(19) United States
(12) Patent Application Publication
(10) Pub. No.: US 2023/0201083 A1
(43) Pub. Date: Jun. 29, 2023

(54) SELF-ADHESIVE DENTAL COMPOSITE CEMENT WITH GOOD TRANSPARENCY BASED ON ACID-TREATED FILLERS

(71) Applicant: Ivoclar Vivadent AG, Schaan (LI)

(72) Inventors: Norbert Moszner, Triesen (LI); Alexandros Gianasmidis, Balgach (CH); Delphine Catel, Rans (CH); Yohann Catel, Rans (CH); Andy Brot, Balzers (LI); Tim Haldner, Mauren (LI); Christian Ritzberger, Grabs (CH); Philipp Bielec, Wasserburg (DE); Marc Dittmer, Feldkirch (AT); Barbara Grabenbauer, Rebstein (CH)

(21) Appl. No.: 18/147,689

(22) Filed: Dec. 28, 2022

(30) Foreign Application Priority Data
Dec. 29, 2021 (EP) 21218194.5

(51) Int. Cl. A61K 6/889; A61K 6/77; A61K 6/79; A61K 6/20

(52) U.S. Cl. CPC A61K 6/889; A61K 6/77; A61K 6/79; A61K 6/20

(57) ABSTRACT

A radically polymerizable dental material having at least one radically polymerizable monomer without acid groups, at least one radically polymerizable monomer containing an acid group, at least one fluoroaluminosilicate glass filler and/or radiopaque glass filler, and at least one initiator for the radical polymerization, wherein the filler is an acid-treated fluoroaluminosilicate glass filler and/or radiopaque glass filler.

### CROSS-REFERENCE TO RELATED APPLICATIONS

This application claims priority to European Patent Application No. 21218194.5 filed on Dec. 29, 2021, the disclosure of which is incorporated herein by reference in its entirety.

### TECHNICAL FIELD

The present invention relates to radically polymerizable, self-adhesive composites with improved transparency which are particularly suitable as dental materials, e.g. as dental cements, filling composites or veneering materials, as well as for the fabrication of inlays, onlays or crowns.

### BACKGROUND

Composites are mainly used in the dental field for the fabrication of direct and indirect fillings, i.e. as direct and indirect filling composites, and as cements. The polymerizable organic matrix of the composites usually consists of a mixture of monomers, initiator components and stabilizers. Mixtures of dimethacrylates are usually used as monomers, which may also contain monofunctional and functionalized monomers. Commonly used dimethacrylates are 2,2-bis[4-(2-hydroxy-3-methacryloyloxypropyl)phenyl]propane (bis-GMA), 1,6-bis[2-methacryloyloxyethoxycarbonylamino]-2,2,4-trimethylhexane (UDMA), which have high viscosity and produce polymers with good mechanical properties and low polymerization shrinkage. Triethylene glycol dimethacrylate (TEGDMA), 1,10-decanediol dimethacrylate (D₃MA) or bis(3-methacryloyloxymethyl)tricyclo-[5.2.1.0^{2.6}]decane (DCP) are mainly used as reactive diluents. Monofunctional methacrylates, such as p-cumylphenoxyethylene glycol methacrylate (CMP-1E), are also suitable for reducing viscosity and cause a reduction in network density and increased double-bond conversion.

To produce self-adhesive composites, strongly acidic adhesive monomers are used, such as 10-methacryloyloxydecyl dihydrogen phosphate (MDP), which etches the tooth structure and causes adhesion to enamel/dentin by ionic relationship. Adhesive monomers impart self-adhesive properties to composites and thus enable the composites to be used without pre-treatment of the tooth structure with an enamel/dentin adhesive, which makes their use particularly attractive.

In addition to the organic matrix, composites contain one or more fillers, which are usually surface-modified with a polymerizable coupling agent, such as 3-methacryloyloxypropyltrimethoxysilane. Fillers improve the mechanical properties (strength, modulus of elasticity, abrasion resistance) and the processing properties (paste consistency, sculptability) of the materials and impart radiopacity.

It is problematic that acidic adhesive monomers often interact adversely with fillers. For example, the acidic adhesion monomers are bound to the surface of the fillers by the formation of insoluble salts, or they form poorly soluble salts during storage with ions released from the fillers. This leads to a significant reduction of the adhesion monomer concentration in the resin matrix, which is associated with a reduction or even a loss of the adhesion properties. Composites with acidic adhesive monomers therefore have only limited storage stability.

Methacrylate-based dental materials are cured by radical polymerization, using radical photoinitiators, thermal initiators or redox initiator systems, depending on the field of application. Dual-curing systems contain a combination of photoinitiators and redox initiators.

Composite cements usually contain redox systems because they ensure sufficient curing even when light curing is not possible due to insufficient transmittance. Redox initiator systems based on a mixture of dibenzoyl peroxide (DBPO) with tertiary aromatic amines, such as N,N-diethanol-p-toluidine (DEPT), N,N-dimethyl-sym.-xylidine (DMSX) or N,N-diethyl-3,5-di-tert-butylaniline (DABA), are typically used. Since radical formation in DBPO/amine-based redox initiator systems is greatly impaired by strong acids and thus also by strongly acidic adhesive monomers, redox initiator systems containing cumene hydroperoxide in combination with thioureas, such as acetylthiourea, are preferred.

In order to ensure sufficient storage stability of the redox initiators, redox initiator system-based materials are usually used as so-called 2-component systems (2C), whereby the oxidizing agent (peroxide or hydroperoxide) and the reducing agent (amines, sulfinic acids, barbiturates, thioureas, etc.) are incorporated into separate components. These are mixed together just before use. For mixing, double-push syringes are preferably used which have separate cylindrical chambers to hold the components. The components are pushed out of the chambers simultaneously by two interconnected pistons and mixed together in a nozzle. To obtain mixtures that are as homogeneous as possible, it is advantageous to mix the components together in approximately equal volume proportions.

Conventional luting cements, such as ZnO eugenol cements, zinc phosphate cements, glass ionomer cements (GIC) and resin modified glass ionomer cements (RMGI), are not suitable for use with double-push syringes because they contain a powder component, which makes mixing of the components considerably more difficult. In addition, glass ionomer cements have only low transparency and relatively poor mechanical properties.

Conventional glass ionomer cements (GIC) contain an aqueous solution of a high molecular weight polyacrylic acid (PAA, number average molar mass greater than 30,000 g/mol) or a copolymer of comparable molar mass of acrylic acid and itaconic acid as liquid component and a calcium fluorine aluminum glass as powder component. After mixing the components, they cure by purely ionic ionomer formation. The disadvantages of glass ionomer cements are their low transparency and poor mechanical properties.

Resin modified glass ionomer cements (RMGI) contain additional hydrophilic monomers, such as 2-hydroxyethyl-methacrylate (HEMA). They cure both by an acid-base reaction and by radical polymerization. Compared to conventional GIC, they are characterized by improved flexural strength.

U.S. Pat. No. 8,053,490 B2, which is hereby incorporated by reference in its entirety, discloses fluoride-releasing dental materials containing a fluoroaluminosilicate glass filler (FAS) which has been reacted with an aqueous solution of a monomer or oligomer containing an acid group. The monomer or oligomer containing an acid group is bound to the filler surface and is said to prevent a reaction of the filler with reactive components of the cement. The materials are characterized by low transparency and unsatisfactory mechanical properties.

JP 2016030741 A1 discloses dental materials containing as a filler a fluoroboroaluminosilicate glass surface-modified with a silane and a polymeric carboxylic acid. The fillers have a spherical structure and are said to exhibit sustained release of fluoride ions and other ions. The dental materials do not exhibit self-adhesion to dentin and enamel and have only unsatisfactory mechanical properties.

US 2016/0324729 A1, which is hereby incorporated by reference in its entirety, discloses fillers for glass ionomer cements whose surface is first silanized and then modified with an unsaturated carboxylic acid, which is bonded to the filler surface via a silicon atom. The carboxy group of the carboxylic acid is said to interact with the constituents of the glass ionomer cement and thus stabilize the cement. The materials have only moderate transparency and poor mechanical properties.

CN 102976618 A discloses low-cost glass fillers for water-based glass ionomer cements containing 25-35 wt. % A₂IO₃, 30-45 wt. % SiO₂, 2-8 wt. % Na₂O, 5-15 wt. % CaF₂ and 5-8 wt. % SrO and/or CaO. The glass powders are treated with hydrochloric acid or acetic acid at room temperature and then dried. They are said to have stable quality and good mechanical properties and continuously release fluoride ions. The glass ionomer cements have low transparency and poor mechanical properties.

Dai et al, Int. J. Nanomedicine 14 (2019) 9185, have shown that surface treatment of basic ZrO₂ fillers with 10-methacryloyloxydecyl dihydrogen phosphate (MDP) can improve the flexural strength of dental composites and that prior coating of ZrO₂ particles with Zr(OH)₄ improves the bonding of MDP to zirconia. Dental materials based on the surface-treated fillers are not self-adhesive and have low transparency.

According to U.S. Pat. No. 8,071,662 B2, which is hereby incorporated by reference in its entirety, surface modification of basic fillers, such as ZrO₂, with a strong acid, such as 3-methacryloyloxypropylsulfonic acid, is said to improve the storage stability of self-etching dental materials. The disclosed dental materials are not self-etching and have unsatisfactory transparency.

DE 24 46 546 A1 discloses the treatment of naturally occurring silica with a hydrous mineral acid to remove acid-soluble impurities. The silica is said to be suitable as a filler for dental purposes. Radiopaque or self-adhesive dental materials are not disclosed.

US 2001/0034309 A1, which is hereby incorporated by reference in its entirety, discloses the treatment of inorganic fillers with peracids or peracid salts to remove organic substances, e. g. carbon. The removal of organic substances is intended to improve the bonding of silane coupling agents to the filler surface and thus the incorporation of the fillers into the organic matrix of radically polymerizable dental materials. Self-adhesive materials are not disclosed.

### SUMMARY

It is an object of the present invention to provide storage-stable, self-adhesive dental composites with good transparency and good mechanical properties, which can be mixed and applied well as 2-component systems using double-push syringes. The composites should be particularly suitable as dental luting cements and be radiopaque.

This object is achieved by filler-containing, radically polymerizable compositions comprising at least one radically polymerizable monomer, at least one acidic, radically polymerizable monomer, at least one fluoroaluminosilicate glass filler and/or radiopaque glass filler, and at least one initiator for the radical polymerization. The compositions are characterized in that the filler has been pre-treated with acid. It was surprisingly found that the acid treatment of the filler causes a significant increase in the storage stability of the compositions.

### BRIEF DESCRIPTION OF THE DRAWINGS

Further details, advantages and features will be apparent from the following description of several exemplary embodiments of the invention with reference to the drawings, in which:

FIG. 1 shows a decrease in concentration of the acidic monomer MDP as a function of storage time in composite pastes with an acid-treated ( [symbol] ) or a non-acid-treated glass filler ( [symbol] ); and

FIG. 2 shows a decrease in concentration of the acidic monomer MDP as a function of storage time in composite pastes with an acid-treated ( [symbol] ) or a non-acid-treated glass filler ( [symbol] ).

### DETAILED DESCRIPTION

The compositions according to the invention contain at least one fluoroaluminosilicate glass filler (FAS filler) and/or a radiopaque glass filler. Compositions containing fillers are referred to as composites.

Preferred radiopaque glass fillers have the following composition (wt. %): SiO₂: 20-80; B₂O₃: 2-15; BaO or SrO: 0-40; Al₂O₃: 2-20; CaO and/or MgO: 0-20; Na₂O, K₂O, Cs₂O: 0-10 each; WO₃: 0-20; ZnO: 0-20; La₂O₃: 0-10; ZrO₂: 0-15; P₂O₅: 0-30; Ta₂O₅, Nb₂O₅ or Yb₂O₃: 0-5; and CaF₂ or SrF₂: 0-10. Particularly preferred are radiopaque glass fillers with the composition (wt. %): SiO₂: 50-75; B₂O₃: 2-15; BaO or SrO: 2-35; Al₂O₃: 2-15; CaO and/or MgO: 0-10 and Na₂O: 0-10.

Particularly preferred FAS fillers have the following composition (wt. %): SiO₂: 20-35; Al₂O₃: 15-35; BaO or SrO: 10-25; CaO: 0-20; ZnO: 0-15; P₂O₅: 5-20; Na₂O, K₂O, Cs₂O: 0-10 each; and CaF₂: 0.5-20. Particularly preferred are FAS fillers with the composition (wt. %): SiO₂: 20-30; Al₂O₃: 20-30; BaO or SrO: 10-25; CaO: 5-20; P₂O₅: 5-20; Na₂O: 0-10; and CaF₂: 5-20.

All percentages are based on the total mass of the glass with the components, except fluorine, being calculated as oxides, as is usual for glasses and glass ceramics.

The compositions according to the invention preferably contain 10 to 80 wt. %, more preferably 20 to 75 wt. %, and most preferably 30 to 70 wt. % of FAS filler and/or radiopaque glass filler, in each case based on the total mass of the composition.

The FAS fillers and radiopaque glass fillers preferably have an average particle size of 0.2 to 20 μm and particularly preferably of 0.4 to 5 μm.

Unless otherwise indicated, all particle sizes herein are volume averaged particle sizes (D50 values), i. e. 50% of the total volume of all particles is contained in particles having a diameter smaller than the indicated value.

Particle size determination in the range from 0.1 μm to 1,000 μm is preferably carried out by means of static light scattering (SLS), for example with an LA-960 static laser scattering particle size analyzer (Horiba, Japan) or with a Microtrac S100 particle size analyzer (Microtrac, USA). Here, a laser diode with a wavelength of 655 nm and an LED with a wavelength of 405 nm are used as light sources. The use of two light sources with different wavelengths enables the measurement of the entire particle size distribution of a sample in only one measurement operation, whereby the measurement is carried out as a wet measurement. For this purpose, an aqueous dispersion of the filler is prepared and its scattered light is measured in a flow cell. The scattered light analysis for calculating particle size and particle size distribution is carried out according to the Mie theory according to DIN/ISO 13320. The measurement of the particle size in a range from 1 nm to 0.1 μm is preferably carried out by dynamic light scattering (DLS) of aqueous particle dispersions, preferably with a He—Ne laser with a wavelength of 633 nm, at a scattering angle of 90° and at 25° C., e.g. with a Malvern Zetasizer Nano ZS (Malvern Instruments, Malvern UK).

In the case of aggregated and agglomerated particles, the primary particle size can be determined by means of TEM images. Transmission electron microscopy (TEM) is preferably performed using a Philips CM30 TEM at an accelerating voltage of 300 kV. For sample preparation, drops of particle dispersion are applied to a 50 Å thick copper grid (mesh size 300 mesh) coated with carbon, followed by evaporation of the solvent. The particles are counted and the arithmetic mean is calculated.

The acid treatment of the particulate FAS or glass fillers is carried out by washing the filler with acid, preferably by the following process:
(i) The particulate FAS or glass filler is dispersed in an aqueous solution of an organic or preferably inorganic acid. The acid is preferably used in a concentration of 0.1 to 5 mol/l, particularly preferably 0.5 to 3 mol/l.
(ii) The dispersion from step (i) is then stirred, preferably for 0.5 to 24 h, more preferably for 1 to 5 h.
(iii) After the stirring in an acid solution, the filler is separated and washed with deionized water. For this purpose, it is preferably dispersed in water and stirred for 1 to 60 minutes, particularly preferably for 2 to 20 minutes.
(iv) After washing, the filler is separated and dried, preferably in a vacuum drying oven in a fine vacuum at 20 to 80° C., particularly preferably at 40 to 60° C. The filler is preferably dried to constant weight.
(v) After drying, the filler is preferably subjected to an optional thermal treatment.

For this purpose, it is preferably heated for 2 to 12 h, particularly preferably for 4 to 6 h, to a temperature that is well below the glass transition temperature of the filler, preferably 200 to 500° C. and particularly preferably 300 to 400° C.

In the process according to the invention, the acid used to treat the fillers is preferably completely removed from the fillers after the acid treatment. The fillers are not coated with an acid.

The acid treatment can be repeated once or several times. Steps (i) and (iii) are preferably carried out at a temperature in the range from 5 to 50° C., particularly preferably at room temperature (23° C.). The temperature is measured in the solution or dispersion in each case.

Acids that form soluble salts with Ca, Al, Sr and Ba ions are preferred. Particularly preferred are formic acid, acetic acid and especially hydrochloric acid, and nitric acid. Alternatively, acids that form sparingly soluble salts with Ca, Al, Sr or Ba ions, such as phosphoric acid, may be used but these are less preferred. Poorly soluble salts are defined as salts with a solubility of less than 0.1 g/l (in water at room temperature). According to the invention, acidic organic monomers and acidic organic polymers as well as peracids and hydrofluoric acid are not suitable as acids.

The washing step (iii) is preferably repeated 1 to 5 times, particularly preferably 3 times, so that the acid is completely removed from the filler. For this purpose, the filler is separated from the water following step (iii) and again dispersed and stirred in deionized water. The washing is repeated until the pH of the water in the last washing step is ≥5.

After washing, the filler can be subjected to further acid treatment and washing. The sequence of acid treatment and washing can be repeated once or several times.

After drying and the optional thermal treatment, the fillers according to the invention are preferably surface-modified, particularly preferably silanized. The silanization is preferably carried out with a radically polymerizable silane, in particular with e.g. 3-methacryloyloxypropyltrimethoxysilane (MEMO). The fillers are readily miscible with the other constituents of the composite materials.

Surprisingly, it was found that acid treatment of the fillers significantly improves the storage stability of the compositions according to the invention, and that the content of radical polymerizable monomers containing an acid group decreases only slowly. The compositions show high adhesion to the tooth structure and particularly to dentin even after prolonged storage. The acid treatment thus enables a significant improvement in the properties of dental composites with self-adhesive properties in a simple manner. After curing, the compositions according to the invention are also characterized by high transparency compared to glass ionomer cements.

In addition to the aforementioned FAS and radiopaque glass fillers, the compositions according to the invention may contain further fillers.

Preferred further fillers are metal oxides, particularly preferably mixed oxides, which contain 60 to 80 wt. % SiO₂ and at least one of the metal oxides ZrO₂, Yb₂O₃, ZnO, Ta₂O₅, Nb₂O₅ and/or La₂O₃, preferably ZrO₂, Yb₂O₃ and/or ZnO, so that the total amount adds up to 100%. Mixed oxides such as SiO₂—ZrO₂ are accessible, e.g. by hydrolytic co-condensation of metal alkoxides. The metal oxides preferably have an average particle size of 0.05 to 10 μm and particularly preferably of 0.1 to 5 μm. The metal oxide or oxides are preferably also treated with acid in the manner described above.

Other preferred additional fillers are pyrogenic silica or precipitated silica with a primary particle size of 0.01 to 0.15 μm, as well as quartz or glass ceramic powder with a particle size of 0.1 to 15 μm, preferably from 0.2 to 5 μm, and ytterbium trifluoride. The ytterbium trifluoride preferably has a particle size of 80 to 900 nm and particularly preferably of 100 to 300 nm. These fillers are preferably used in an amount of 0.1 to 25 wt. %, more preferably 0.2 to 20 wt. %, and most preferably 0.3 to 15 wt. %, in each case based on the total mass of the composition.

In addition, so-called composite fillers are preferred as further fillers. These are also referred to as isofillers. These are splinter-like polymers which, in turn, contain a filler, preferably pyrogenic SiO₂, glass filler and/or ytterbium trifluoride. Polymers based on dimethacrylates are preferred. For the production of isofillers, the filler(s) is/are incorporated, for example, into a dimethacrylate resin matrix, and the resulting composite paste is subsequently thermally polymerized and then ground.

A composite filler preferred according to the invention can be prepared, for example, by thermally curing a mixture of bis-GMA (8.80 wt. %), UDMA (6.60 wt. %), 1,10-decanediol dimethacrylate (5.93 wt. %), dibenzoyl peroxide +2,6-di-tert.-butyl-4-methylphenol (together 0.67 wt. %), glass filler (average grain size 0.4 μm; 53.0 wt. %) and YbF₃ (25.0 wt. %) and then grinding the cured material to the desired grain size. All percentages refer to the total mass of the composite filler.

So-called inertized fillers can also be used as further fillers. These are glass fillers whose surface is coated with a diffusion barrier layer, e.g. on a sol-gel basis, or with a polymer layer, e.g. of PVC. Preferred fillers are those described in EP 2 103 296 A1 and corresponding U.S. Pat. No. 7,932,304 B2, which US patent is hereby incorporated by reference in its entirety.

To improve the bond between filler and matrix, the fillers are preferably surface-modified with methacrylate functionalized silanes, such as 3-methacryloyloxypropyltrimethoxysilane.

The compositions according to the invention preferably contain 0.1 to 25 wt. %, preferably 1 to 20 wt. % and particularly preferably 2 to 15 wt. % of one or more further fillers, preferably one or more metal oxides, pyrogenic silica and/or precipitated silica, in each case based on the total mass of the composition.

The compositions according to the invention contain at least one radically polymerizable monomer, preferably one or more mono- and/or polyfunctional monomers. Polyfunctional monomers are understood to be compounds with two or more, preferably 2 to 4 and particularly preferably 2, radically polymerizable groups. Accordingly, monofunctional monomers have only one radically polymerizable group. Polyfunctional monomers have crosslinking properties and are therefore also referred to as crosslinking monomers. Preferred radically polymerizable groups are (meth)acrylate, (meth)acrylamide, and vinyl groups.

According to the invention, a distinction is made between monomers containing acid groups and monomers which do not contain acid groups. The compositions according to the invention contain at least one monomer without acid groups and at least one monomer and/or oligomer with acid groups. The compositions according to the invention preferably comprise monomers with and monomers without acid groups in a weight ratio of from 1:5 to 1:36, more preferably from 1:6 to 1:25, and most preferably from 1:7 to 1:20.

### Monomers Without Acid Group

Preferred are compositions comprising at least one (meth)acrylate, more preferably at least one monofunctional or polyfunctional methacrylate, and most preferably at least one monofunctional or difunctional methacrylate, or a mixture thereof.

Preferred monofunctional (meth)acrylates are benzyl, tetrahydrofurfuryl or isobornyl (meth)acrylate, p-cumyl phenoxyethylene glycol methacrylate (CMP-1E) and 2-([1,1′-biphenyl]-2-oxy)ethyl methacrylate (MA-836), tricyclodecane methyl (meth)acrylate, 2-(2-biphenyloxy)ethyl (meth)acrylate. CMP-1E and MA-836 are particularly preferred.

According to one embodiment, the compositions according to the invention preferably comprise at least one functionalized monofunctional (meth)acrylate. Functionalized monomers are understood to be those monomers which, in addition to at least one radically polymerizable group, carry at least one functional group, preferably a hydroxyl group. Preferred functionalized mono(meth)acrylates are 2-hydroxyethyl and hydroxyethyl propyl(methacrylate) and 2-acetoxyethyl methacrylate. Hydroxyethyl methacrylate is particularly preferred. The monomers containing acid groups mentioned below are not functionalized monomers within the meaning of the invention.

Preferred di- and polyfunctional (meth)acrylates are bisphenol-A-dimethacrylate, bis-GMA (an addition product of methacrylic acid and bisphenol-A-diglycidyl ether), ethoxy- or propoxylated bisphenol-A-dimethacrylates, e.g. the bisphenol A dimethacrylate SR-348c (Sartomer) with 3 ethoxy groups or 2,2-bis[4-(2-methacryloyloxypropoxy)phenyl]propane, urethanes of 2-(hydroxymethyl)acrylic acid methyl ester and diisocyanates, such as 2,2,4-trimethylhexamethylene diisocyanate or isophorone diisocyanate, UDMA (an addition product of 2-hydroxyethyl methacrylate- and 2,2,4-trimethylhexamethylene-1,6-diisocyanate), tetramethylxylylene diurethane ethylene glycol di(meth)acrylate or tetramethylxylylene diurethane-2-methylethylene glycol di(meth)acrylate (V380), di-, tri- or tetraethylene glycol dimethacrylate, trimethylolpropane trimethacrylate, pentaerythritol tetramethacrylate as well as glycerol di- and -trimethacrylate, 1,4-butanediol dimethacrylate, 1,10-decanediol dimethacrylate (D₃MA), bis(methacryloyloxymethyl)tricyclo-[5.2.1.0^{2.6}]decane (DCP), polyethylene glycol or polypropylene glycol dimethacrylates, such as polyethylene glycol 200-dimethacrylate or polyethylene glycol 400-dimethacrylate (PEG-200- or PEG-400-DMA) or 1,12-dodecanediol dimethacrylate. Bis-GMA, UDMA, V-380, triethylene glycol dimethacrylate (TEGDMA) and PEG-400-DMA (NK ester 9G) are particularly preferred.

The monomer tetramethylxylylene diurethane ethylene glycol di(meth)acrylate or tetramethylxylylene diurethane 2-methylethylene glycol diurethane di(meth)acrylate (V380) has the following formula:

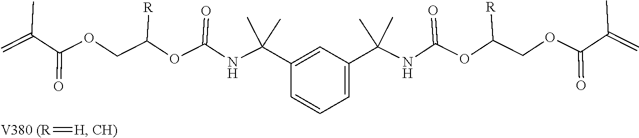

In the formula shown, the radicals R are each independently H or CH₃, and the radicals may have the same meaning or different meanings. Preferably, a mixture is used which contains molecules in which both radicals are H, molecules in which both radicals are CH₃, and molecules in which one radical is H and the other radical is CH₃, with a ratio of H to CH₃ of 7:3 being preferred. Such a mixture is obtainable, for example, by reacting 1,3-bis(1-isocyanato-1-methylethyl)benzene with 2-hydroxypropyl methacrylate and 2-hydroxyethyl methacrylate.

Other preferred difunctional monomers include radically polymerizable pyrrolidones, such as 1,6-bis(3-vinyl-2-pyrrolidonyl)-hexane, or commercially available bisacrylamides such as methylene or ethylene bisacrylamide, as well as bis(meth)acrylamides, such as N,N′-diethyl-1,3-bis(acrylamido)propane, 1,3-bis(methacrylamido)propane, 1,4-bis(acrylamido)butane or 1,4-bis(acryloyl)piperazine, which can be synthesized from the corresponding diamines by reaction with (meth)acrylic acid chloride. N,N′-diethyl-1,3-bis(acrylamido)propane (V-392) is particularly preferred. These monomers are characterized by high hydrolytic stability.

### Monomers and Oligomers Containing Acid Groups

The compositions according to the invention contain at least one acidic radically polymerizable monomer and/or at least one acidic oligomer. Acidic monomers and oligomers are understood to mean monomers and oligomers, respectively, which contain at least one acid group, preferably a phosphoric ester, phosphonic acid or carboxy group. Acidic monomers and oligomers are also referred to herein as adhesive components or adhesive monomers or adhesive oligomers. In accordance with the invention, those compositions which contain at least one strongly acidic monomer are particularly preferred. Strongly acidic monomers are monomers with a pKa value of 0.5 to 4.0, more preferably 1.0 to 3.5, and most preferably 1.5 to 2.5 at room temperature.

Suitable monomers containing acid groups are COOH group-containing polymerizable monomers, preferably with a pKa value in the range of 2.0 to 4.0. 4-(Meth)acryloyloxyethyltrimellitic anhydride, 10-methacryloyloxydecylmalonic acid, N-(2-hydroxy-3-methacryloyloxypropyl)-N-phenylglycine and 4-vinylbenzoic acid are preferred. Methacrylic acid (pKa=4.66) is excluded due to its low adhesion to the tooth structure.

Preferred monomers containing acid groups are phosphoric ester and phosphonic acid monomers, preferably with a pKa value in the range of 0.5 to 3.5. Particularly preferred are 2-methacryloyloxyethylphenyl hydrogen phosphate, 10-methacryloyloxydecyl dihydrogen phosphate (MDP) glycerol dimethacrylate dihydrogen phosphate or dipentaerythritol pentamethacryloyloxy phosphate, 4-Vinylbenzylphosphonic acid, 2-[4-(dihydroxyphosphoryl)-2-oxa-butyl]-acrylic acid or hydrolysis-stable esters, such as 2-[4-(dihydroxyphosphoryl)-2-oxa-butyl]acrylic acid 2,4,6-trimethylphenyl ester. MDP, 2-methacryloyloxyethylphenyl hydrogen phosphate and glycerol dimethacrylate dihydrogen phosphate are even more preferred.

Oligomers are understood to be polymers with a degree of polymerization Pₙ of 2 to 100 (Pₙ=Mₙ/Mᵤ; Mₙ: number average polymer molecular weight, Mᵤ: molecular weight of the monomer unit). Acidic radically polymerizable oligomers have at least one acid group, preferably a carboxyl group, and at least one radically polymerizable group, preferably at least one (meth)acrylate group and in particular at least one methacrylate group.

Oligomers containing acid groups preferred according to the invention are oligomeric carboxylic acids, such as polyacrylic acid, preferably with a number average molecular weight Mₙ of less than 7,200 g/mol, more preferably less than 7,000 g/mol and most preferably less than 6,800 g/mol, where Mₙ is preferably in a range from 800 to 7,200 g/mol, more preferably from 500 to 7,000 g/mol and most preferably from 500 to 6,800 g/mol. Oligomeric carboxylic acids containing (meth)acrylate groups are particularly preferred. These can be obtained, for example, by reacting oligomeric polyacrylic acid with glycidyl methacrylate or 2-isocyanatoethyl methacrylate.

Unless otherwise stated, the molar mass of oligomers and polymers herein is the number-average molar mass, the absolute values of which can be determined by the known methods of freezing point depression (cryoscopy), boiling point elevation (e-bullioscopy), or through the depression of the vapor pressure (vapor pressure osmometry). Preferably, the number average molecular weight of oligomers and polymers is determined by gel permeation chromatography (GPC). This is a relative method in which molecules are separated on the basis of their size, more precisely on the basis of their hydrodynamic volume. The absolute molar mass is determined by calibration with known standards.

The compositions according to the invention also preferably contain water. It has been found that a water content of 1 to 7 wt. %, preferably 1 to 5 wt. %, in each case based on the total mass of the composition, results in an improvement of the bonding effect to dentin and enamel.

The compositions according to the invention further comprise at least one initiator for initiating the radical polymerization, preferably a photoinitiator. Preferred photoinitiators are benzophenone, benzoin, and derivatives thereof, α-diketones or derivatives thereof, such as 9,10-phenanthrenequinone, 1-phenyl-propane-1,2-dione, diacetyl and 4,4′-dichlorobenzil. Particularly preferred are camphorquinone (CQ) and 2,2-dimethoxy-2-phenyl-acetophenone, and most preferred are α-diketones in combination with amines as reducing agents, such as 4-(dimethylamino)-benzoic acid ethyl ester (EDMAB), N,N-dimethylaminoethyl methacrylate, N,N-dimethyl-sym.-xylidine or triethanolamine. Further preferred are Norrish type I photoinitiators, especially acyl or bisacyl phosphine oxides and most preferably monoacyltrialkylgermanium, diacyldialkylgermanium and tetraacylgermanium compounds, such as benzoyltrimethylgerman, dibenzoyldiethylgerman, bis(4-methoxybenzoyl)diethylgerman (Ivocerin®), tetrabenzoylgerman or tetrakis(o-methylbenzoyl)german. Mixtures of the various photoinitiators can also be used, such as bis(4-methoxybenzoyl)diethylgerman or tetrakis(o-methylbenzoyl)german in combination with camphorquinone and 4-dimethylaminobenzoic acid ethyl ester.

Further preferred are compositions containing a redox initiator for initiating radical polymerization, preferably a redox initiator based on an oxidizing agent and a reducing agent. Preferred oxidizing agents are peroxides and in particular hydroperoxides. A particularly preferred peroxide is benzoyl peroxide. Preferred hydroperoxides are the low-odor cumene hydroperoxide derivatives disclosed in EP 3 692 976 A1 and corresponding U.S. Pat. No. 11,357,709 B2, which US patent is hereby incorporated by reference in its entirety, the oligomeric CHP derivatives disclosed in EP 21315089.9, and, in particular, 4-(2-hydroperoxypropan-2-yl)phenylpropionate and cumene hydroperoxide (CHP).

Preferred reducing agents for combination with peroxides are tertiary amines, such as N,N-dimethyl-p-toluidine, N,N-dihydroxyethyl-p-toluidine, p-dimethylaminobenzoic acid ethyl ester or other aromatic dialkylamines, ascorbic acid, sulfinic acids, thiols and/or hydrogen silanes.

Preferred reducing agents for combination with hydroperoxides are thiourea derivatives, in particular the compounds listed in paragraph [0009] of EP 1 754 465 A1 and corresponding US 2007040151 A1, which US published application is hereby incorporated by reference in its entirety. Particularly preferred are methyl-, ethyl-, allyl-, butyl-, hexyl-, octyl-, benzyl-, 1,1,3-trimethyl-, 1,1-diallyl-, 1,3-diallyl-, 1-(2-pyridyl)-2-thiourea, acetyl-, propanoyl-, butanoyl-, pentanoyl-, hexanoyl-, heptanoyl-, octanoyl-, nonanoyl-, decanoyl-, benzoylthiourea and mixtures thereof. Quite particularly preferred are acetyl-, allyl-, pyridyl- and phenylthiourea as well as hexanoylthiourea and mixtures thereof as well as polymerizable thiourea derivatives, such as N-(2-methacryloyloxyethoxysuccinoyl)-thiourea and N-(4-vinylbenzoyl)-thiourea). In addition, a combination of one or more of said thiourea derivatives with one or more imidazoles may advantageously be used. Preferred imidazoles are 2-mercapto-1-methylimidazole or 2-mercaptobenzimidazole.

In addition to at least one hydroperoxide and at least one thiourea derivative, the compositions according to the invention may further comprise at least one transition metal compound for accelerating curing. Transition metal compounds that are suitable according to the invention are, in particular, compounds derived from transition metals with at least two stable oxidation states. Particularly preferred are compounds of the elements copper, iron, cobalt, nickel and manganese. These metals have the following stable oxidation states: Cu(I)/Cu(II), Fe(II)/Fe(III), Co(II)/Co(III), Ni(II)/Ni(III), Mn(II)/Mn(III). Compositions containing at least one copper compound are particularly preferred. The transition metal compounds are preferably used in catalytic amounts, particularly preferably in an amount of 10 to 200 ppm. These amounts do not lead to discoloration of the dental materials. Because of their good monomer solubility, the transition metals are preferably used in the form of their acetylacetonates, 2-ethylhexanoates or THF adducts. Further preferred are their complexes with polydentate ligands such as 2-(2-aminoethylamino)ethanol, triethylenetetramine, dimethylglyoxime, 8-hydroxyquinoline, 2,2′-bipyridine or 1,10-phenanthroline. According to the invention, a particularly preferred initiator is a mixture of cumene hydroperoxide (CHP) with at least one of the above-mentioned thiourea derivatives and copper(II) acetylacetonate. According to the invention, compositions which do not contain vanadium compounds are preferred.

The compositions according to the invention preferably do not contain barbituric acid or barbituric acid derivatives such as 1,3,5-trimethylbarbituric acid, 1-benzyl-5-phenylbarbituric acid, 5-butylbarbituric acid or 1-cyclohexyl-5-ethylbarbituric acid. Compositions containing barbiturates have unsatisfactory storage stability because barbiturates form polymerization-initiating radicals by oxidation with atmospheric oxygen. In addition, barbiturates have adverse physiological effects such as bradycardia, hypotension, or blood disorders.

According to a preferred embodiment, the compositions according to the invention preferably additionally contain a masking agent. According to the invention, preferred masking agents are ethylenediaminetetraacetic acid (EDTA) and its disodium salt (disodium ethylenediaminetetraacetate), nitrilotriacetic acid, diethylenetriaminepentaacetic acid, tetrasodium iminodisuccinate, and the trisodium salt of methylglycinediacetic acid. EDTA is particularly preferred. Further included are polymerizable derivatives of the aforementioned masking agents. Polymerizable derivatives are masking agents with radically polymerizable groups. Preferred radically polymerizable masking agents are EDTA derivatives carrying polymerizable (meth)acrylic or methacrylamide groups. Particularly preferred are the polymerizable EDTA derivatives disclosed in DE 10 2005 022 172 A1, in which EDTA is covalently linked to ethylenically unsaturated monomers, and the alkylenediamine-N,N,N′,N′-tetraacetic acid (meth)acrylamides disclosed in EP 2 065 363 A1 and corresponding US 2009139433 A1, which US published application is hereby incorporated by reference in its entirety. The masking agent may be in dissolved or preferably undissolved form.

The one or more masking agents are preferably added in a total amount of 0.5 to 6.0 wt. %, more preferably 0.7 to 5 wt. %, and most preferably 1.0 to 4.0 wt. %. Unless otherwise stated, all percentages herein refer to the total mass of the composition.

The compositions according to the invention may also contain additional additives, in particular stabilizers, colorants, phase transfer catalysts, microbicidal agents, fluoride ion-releasing additives, such as fluoride salts, in particular NaF or ammonium fluoride, or fluorosilanes, optical brighteners, plasticizers and/or UV absorbers.

According to the invention, compositions which comprise the following ingredients are particularly preferred:
a) 10 to 80 wt. %, preferably 20 to 75 wt. %, and particularly preferably 30 to 70 wt. % of at least one acid-treated FAS and/or glass filler,
b) optionally 0.1 to 25 wt. %, preferably 1 to 20 wt. %, and particularly preferably 2 to 15 wt. % of one or more further fillers,
c) 1 to 15 wt. %, preferably 2 to 12 wt. % and particularly preferably 3 to 10 wt. % of at least one monomer containing an acid group,
d) 5 to 40 wt. %, preferably 8 to 30 wt. %, particularly preferably 10 to 25 wt. % of at least one polyfunctional monomer without acid groups,
e) 0 to 10 wt. %, preferably 0 to 8 wt. % and particularly preferably 1 to 5 wt. % of one or more oligomeric carboxylic acids,
f) 1 to 20 wt. %, preferably 2 to 15 wt. %, and particularly preferably 3 to 10 wt. % of one or more monofunctional monomers without acid groups,
g) 0.1 to 8 wt. %, preferably 0.5 to 6 wt. %, and particularly preferably 1 to 5 wt. % of an initiator for radical polymerization,
h) 0 to 20 wt. %, preferably 0.2 to 10 wt. %, and particularly preferably 1 to 7 wt. % of water, and
i) 0.01 to 5 wt. %, preferably 0.1 to 3 wt. %, and particularly preferably 0.1 to 2 wt. % of one or more additives, in each case based on the total mass of the composition.

The initiator can be a redox initiator, a photoinitiator, or an initiator for dual curing. The amounts mentioned include all initiator components, i.e. the initiators themselves and, if present, reducing agents, transition metal compound etc. According to the invention, compositions containing at least one redox initiator or at least one redox initiator and at least one photoinitiator are preferred.

Compositions containing a redox initiator are also referred to as self-curing. They are preferably used in the form of two spatially separated components, i.e. as a 2-component system (2C system). Oxidizing and reducing agents are incorporated into separate components of the composition. One component, the so-called catalyst paste, contains the oxidizing agent, preferably a peroxide or hydroperoxide. The second component, the so-called base paste, contains the corresponding reducing agent and optionally a photoinitiator and optionally catalytic amounts of a transition metal compound. Polymerization is initiated by mixing the components. Compositions containing both a redox initiator and a photoinitiator are referred to as dual-curing.

If the compositions contain a masking agent, it is preferably added to the component in which the strongly acidic adhesive monomer, the FAS filler and/or the radiopaque glass filler are located.

According to the invention, 2-component systems are preferred. They are preferably self-curing or dual-curing. The pastes are mixed together shortly before use, preferably with a double-push syringe.

The catalyst paste preferably has the following composition:
a) 10 to 80 wt. %, preferably 20 to 75 wt. %, and particularly preferably 30 to 70 wt. % of at least one acid-treated FAS and/or glass filler,
b) optionally 0.1 to 25 wt. %, preferably 1 to 20 wt. %, and particularly preferably 2 to 15 wt. % of one or more further fillers,
c) 2 to 30 wt. %, preferably 4 to 24 wt. %, and particularly preferably 6 to 20 wt. % of at least one monomer containing an acid group,
d) 5 to 40 wt. %, preferably 8 to 30 wt. %, particularly preferably 10 to 25 wt. % of at least one polyfunctional monomer without acid groups,
e) 0 to 10 wt. %, preferably 0 to 8 wt. %, and particularly preferably 1 to 5 wt. % of one or more oligomeric carboxylic acids,
f) 1 to 20 wt. %, preferably 2 to 15 wt. %, and particularly preferably 3 to 10 wt. % of one or more monofunctional monomers without acid groups,
g) 0.01 to 16 wt. %, preferably 0.2 to 12 wt. %, and particularly preferably 0.5 to 10 wt. % of at least one peroxide and/or hydroperoxide and optionally at least one photoinitiator,
h) 0 to 20 wt. %, preferably 0.2 to 10 wt. %, and particularly preferably 1 to 7 wt. % of water, and
i) 0.001 to 5 wt. %, preferably 0.002 to 3 wt. %, and particularly preferably 0.0051 to 2 wt. % of one or more additives, in each case based on the total mass of the catalyst paste.

The base paste preferably has the following composition:
a) 10 to 80 wt. %, preferably 20 to 75 wt. %, and particularly preferably 30 to 70 wt. % of at least one acid-treated FAS and/or glass filler,
b) 0.1 to 25 wt. %, preferably 1 to 20 wt. %, and particularly preferably 2 to 15 wt. % of one or more further fillers,
d) 5 to 40 wt. %, preferably 8 to 30 wt. %, particularly preferably 10 to 25 wt. % of at least one polyfunctional monomer without acid groups,
f) 1 to 20 wt. %, preferably 2 to 15 wt. % and particularly preferably 3 to 10 wt. % of one or more monofunctional monomers without acid groups,
g) 0.01 to 16 wt. %, preferably 0.3 to 12 wt. %, and particularly preferably 1 to 10 wt. % of at least one suitable reducing agent and optionally at least one photoinitiator,
h) 0 to 20 wt. %, preferably 0.2 to 10 wt. %, and particularly preferably 1 to 7 wt. % of water, and
i) 0.001 to 5 wt. %, preferably 0.002 to 3 wt. %, and particularly preferably 0.0051 to 2 wt. % of one or more additives, in each case based on the total mass of the base paste.

For application, the catalyst and base paste are preferably mixed together in approximately equal proportions. They are therefore particularly suitable for application with a double-push syringe.

Double-push syringes have two separate cylindrical chambers for holding the base paste and the catalyst paste. The components are pressed out of the chambers simultaneously by two interconnected pistons and are preferably forced through a mixing cannula and mixed together therein. For pressing out the pastes, the syringe can be inserted into a so-called hand dispenser, which facilitates handling of the syringes.

The compositions according to the invention are characterized by high storage stability and improved transparency, preferably greater than 10%, and good self-adhesion to enamel/dentin. They are particularly suitable as dental materials for intraoral use by the dentist for the restoration of damaged teeth (therapeutic use), especially as dental cements, coating or veneering materials, filling composites and most particularly as luting cements. The transparency is determined in the manner described in the examples.

For the treatment of damaged teeth, these are preferably prepared in a first step by the dentist. Subsequently, at least one composition according to the invention is applied to or into the prepared tooth. Thereafter, the composition can be cured directly, preferably by irradiation with light of a suitable wavelength, e.g. when restoring cavities. Alternatively, a dental restoration, e.g. an inlay, onlay, veneer, crown, bridge, framework, or dental ceramic, is placed in or applied to the prepared tooth. Subsequent curing of the composition is preferably done by light and/or self-curing. In this process, the dental restoration is attached to the tooth.

The compositions according to the invention can also be used as extraoral materials (non-therapeutic), e.g. in the fabrication or repair of dental restorations. They are also suitable as materials for the fabrication and repair of inlays, onlays, crowns, or bridges.

For the production of dental restorations such as inlays, onlays, crowns or bridges, at least one composition according to the invention is formed into the desired dental restoration in a manner known per se and then cured. The curing can be done by light, through self-curing, or preferably thermally.

In the repair of dental restorations, the compositions according to the invention are placed onto the restoration to be repaired, for example to repair gaps or to bond fragments, and then cured.

The invention is explained in more detail below with reference to figures and examples.

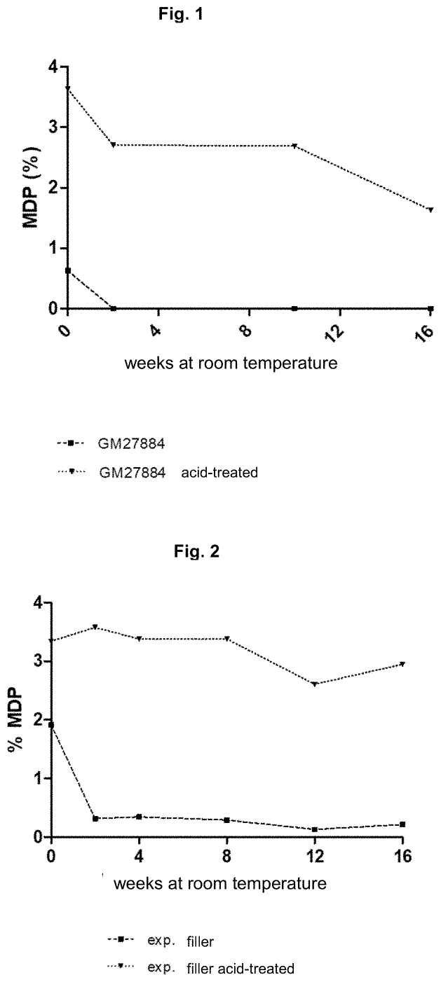

FIG. 1 and FIG. 2 each show the decrease in concentration of the acidic monomer MDP as a function of storage time in composite pastes with an acid-treated ( [symbol] ) or a non-acid-treated glass filler ( [symbol] ).

### Example 1

### Production of Acid-Treated Fillers (General Procedure)

In two centrifuge plastic vessels, each with a filling volume of 1 l, 150 g of the filler to be treated and 350 g of 1.0 molar hydrochloric acid are poured in and stirred for 1 h at room temperature on a magnetic stirring plate. After removing the magnetic stirrer, the mixtures are centrifuged in a centrifuge (Hettich Silenta RS) for 5 min at 3,000 rpm, during which the filler settles. The liquid is separated and a sample of the liquid is taken for X-ray fluorescence (XRF) analysis. The pH of the liquid is 1-2. The separated filler is then dispersed in 400 ml of deionized water and the dispersion is centrifuged again for 5 min at 3,000 rpm. Then, the washing solution is separated by decantation. The washing procedure is repeated until the pH increases to 5 or more during the last washing step (approximately three times). After washing, the acid-treated filler is dried in the vacuum drying oven at 50° C. until constant weight is achieved, and then silanized. For silanization, 12 g of 3-methacryloyloxypropyltrimethoxy-silane (Silane A-174, Sigma Aldrich) was added to the filler (185 g) and then mixed for 15 min (Turbola mixer, Willy A. Bachofen AG). Then, 5 g deionized water was added and mixed again for 15 min. The filler is then sieved through a 90 μm plastic sieve, allowed to rest for 24 h, and then dried in a drying oven at 50° C. for 3 days until no free silane is detectable (gas chromatography).

### Example 2

### Investigation of the Storage Stability of Composites Based on the Filler GM 27884 (With and Without Acid Treatment)

A radiopaque dental glass filler (GM 27884, Schott, mean particle size 1 μm, specific surface area (BET DIN ISO 9277) 3.9 m²/g, composition (wt. %): Al₂O₃: 10, B₂O₃: 10, BaO: 25 and SiO₂: 55) was treated with acid and silanated in the manner described in Example 1. Aluminum and barium ions were primarily detected in the acid treatment solution by XRF analysis. For comparison purposes, a portion of the filler was silanized without prior acid treatment.

Composite pastes were prepared with the acid-treated filler (paste 1) and the untreated filler (paste 2) with the following composition (wt. %): filler: 65.39, 10-methacryloyloxydecyl dihydrogen phosphate (MDP, Orgentis): 3.67, triethylene glycol dimethacrylate (TEGDMA): 9.52, NK ester 9G (polyethylene glycol 400 dimethacrylate, Kowa Europa GmbH): 2.12, V-392 (N,N′-diethyl-1,3-bis(acrylamido)-propane, Ivoclar Vivadent AG): 13.04, BHT (2,6-di-tert-butyl-p-cresol): 0.04, and deionized water: 6.23.

The pastes were stored at room temperature and at intervals of a few weeks the content of MDP was determined by HPLC. For HPLC measurement, an HPLC Ultimate 3000 (ThermoFisher Scientific) instrument with a 125×4 Nucleodur 100-5 C18ec column and a UV/VIS detector (220 nm) was used. The sample was dissolved in methanol and eluted with 0.01 mol/l H₃PO₄ in water (A), methanol (B) and acetonitrile (C) according to the following program. The results are shown in FIG. 1.

### Gradient Program

| Time (min) | % A | % B | % C |
|---|---|---|---|
| 0 | 40% | 40% | 20% |
| 6 | 40% | 40% | 20% |
| 20 | 0% | 0% | 100% |
| 24 | 0% | 0% | 100% |

The results shown in FIG. 1 demonstrate a markedly improved storage stability of the paste based on the acid-treated filler. In the paste with the untreated filler, the MDP was no longer detectable after only 1 week.

### Example 3

### Preparation of Dual-Curing Self-Adhesive Composite Cements

Dual-curing composite cements were prepared. Each cement included a catalyst paste and a base paste. The compositions of the pastes are given in tables 1 and 2. The glass filler used to prepare catalyst paste 1 was treated with acid in the manner described in Example 1. All fillers were silanized. The dentin adhesion was determined as a function of the storage time. For the investigation of dentin adhesion, bovine teeth were embedded in a plastic cylinder with an addition-curing vinyl polysiloxane (Dreve) in such a way that the dentin and the plastic were in one plane. The tooth surfaces were ground with abrasive paper (grit 400) and then were rinsed with lukewarm water, and pre-tempered to 37° C. The dentin surfaces were blotted dry, the catalyst pastes were each mixed with the corresponding base paste in a 1:1 ratio and then applied to the tooth surface. At the same time, the underside of a plug made of a cured dental composite material (Tetric Evo-Ceram, Ivoclar Vivadent AG) was wetted with the cement and placed centrally on the dentin surface. The tooth was then clamped with the composite plug facing upwards in an Ultradent clamping device so that the fixation mandrel was centered on the Tetric Evo-Ceram plug. The excess luting cement was then carefully removed immediately and the Ultradent application device with the clamped tooth was stored for 15 min at 37° C. in a drying cabinet. The plugs were then relieved, kept in water at 37° C. for 24 h, and then stored in the drying cabinet at 37° C. for 24 h. To measure shear bond strength, the plugs were sheared at 23° C. using a Zwick testing machine according to the Ultradent method (EN ISO 29022, 2013), and the shear bond strength was obtained as the quotient of the breaking force and the bond area.

With the cement according to the invention consisting of base paste 1 and catalyst paste 1, an initial dentin bond strength value of 12.0 MPa was determined. After 2 weeks of storage of the pastes at room temperature, the value was 10.3 MPa. In contrast, the cement with the untreated filler consisting of base paste 2 and catalyst paste 2 gave an initial value of only 1.9 MPa, which dropped to a value of 0 MPa after 2 weeks storage at room temperature.

TABLE 1
Composition of the base pastes (data in wt. %)

| Component | Base paste 1 | Base paste 2*) |
|---|---|---|
| TEGDMA | 8.54 | 8.62 |
| Polyethylene glycol 400 dimethacrylate | 2.51 | 2.53 |
| UDMA | 12.50 | 12.62 |
| N,N-diethyl-3,5-di-tert•butyl-aniline | 1.498 | 0.600 |
| Camphorquinone | 0.075 | 0.076 |
| 4-(dimethylamino)benzoic acid ethyl ester | 0.151 | 0.152 |
| Benzyltributylammonium chloride ¹ | 0.201 | 0.203 |
| Color pigment (Lumilux LZ Flu Blue)⁶ | 0.00074 | 0.00074 |
| Inhibitor² | 0.00077 | 0.00077 |
| Water (deionized) | 0.6442 | 0.6501 |
| FAS filler | 69.35 | 69.98 |
| (G018-056 7.0 μm, 5% silan.)³ | | |
| FAS filler | 3.05 | 3.08 |
| (G018-056 1.0 μm, 5% silan.)⁴ | | |
| HDK 2000⁵ | 1.48 | 1.49 |

*)comparative example
¹ phase transfer catalyst
²TEMPO: 2,2,6,6-tetramethylpiperidinyloxyl, CAS number 2564-83-2.
³24 wt. % Al₂O₃, 23 wt. % SiO₂, 16.5 wt. % CaO, 16 wt. % CaF₂, 11.5 wt. % BaO, 8 wt. % P₂O₅, 2 wt. % Na₂O, 5% silane; weight average particle diameter 7 μm (Schott AG, Mainz).
⁴24 wt. % Al₂O₃, 23 wt. % SiO₂, 16.5 wt % CaO, 16 wt. % CaF₂, 11.5 wt. % BaO, 8 wt. % P₂O₅, 2 wt. % Na₂O, 5% silane; weight average particle diameter 1 μm (Schott AG, Mainz).
⁵pyrogenic silica; trimethylsiloxy surface modification; BET surface area (DIN ISO 9277 DIN 66132) unsilanized: approx. 200 m²/g; density (SiO₂; DIN 51757): 2.2 g/cm³; residual silanol content (relative silanol content based on unsilanized silica with approx. 2 SiOH/nm²): 25% (Wacker Chemie AG)
⁶2,5-dihydroxyterephthalic acid diethyl ester (Riedel-de Haën AG)

TABLE 2
Composition of the catalyst pastes (data in wt. %)

| Component | Catalyst paste 1 | Catalyst paste 2*) |
|---|---|---|
| MDP | 3.41 | 3.41 |
| TEGDMA | 10.72 | 10.72 |
| Polyethylene glycol 400 | 2.38 | 2.38 |
| dimethacrylate | | |
| UDMA | 14.68 | 14.68 |
| Dibenzoyl peroxide (50%) | 0.970 | 0.970 |
| BHT | 0.031 | 0.031 |
| Water (deionized) | 0.831 | 0.831 |
| Glass filler GM 27884 | 63.43 | 63.43 |
| 1.0 μm, 5% silane.¹ | | |
| HDK 2000 | 3.54 | 3.54 |

*)comparative example
¹see Example 2

### Example 4

### Examination of the Transparency of the Composite of Example 3

After complete curing, the transparency of composite 1 consisting of catalyst paste 1 and base paste 1 was measured by means of a spectrophotometer (Konika-Minolta Spectrophotometer CM-5) on 1 mm thick test specimens polished to high gloss in transmission. The transparency was 19.2%. The transparency value of composite cement 1 is significantly higher than that of classical glass ionomer cements, such as Vivaglass CEM PL (Ivoclar Vivadent AG) with a transparency value of 6.7%.

### Example 5

### Investigation of the Storage Stability of Composites Based on a Radiopaque Glass Filler (With and Without Acid Treatment)

An experimental X-ray opaque glass filler (mean particle size 3 μm; composition (wt. %): Al₂O₃: 6; B₂O₃: 5; Na₂O: 8; CaO, BaO, K₂O: 2-3 each; CaF₂, MgO: 1 each; and SiO₂: 70) was treated with acid and silanized in the manner described in Example 1. For comparative purposes, portion of the filler was silanized without prior acid treatment. In the acid treatment solution, mainly Al, Ba, Ca, Na and K ions were detected by XRF analysis. Both the acid treated filler and the untreated filler were used to produce composite pastes with the following composition (wt. %): filler: 65.00, MDP: 3.29, TEGDMA: 8.53, NK-ester 9G: 1.90, V-392: 11.68, BHT: 0.03, deionized water: 5.58 and pyrogenic silica HDK 2000 (Wacker Chemie AG): 4.00.

The pastes with the acid-treated filler and with the untreated filler were stored at room temperature and, at intervals of a few weeks, the MDP content was determined by HPLC analogous to Example 2. The results are shown in FIG. 2. The results shown in FIG. 2 demonstrate a markedly improved storage stability of the paste based on the acid-treated filler. In the paste with the untreated filler, a clear decrease of the MDP content was observed after 2 weeks, while in the paste with the acid treated filler, no decrease of the MDP content was observed even after 8 weeks. The results show that the acid treatment of the filler significantly improves the storage stability.

1. A radical polymerizable composition comprising
at least one radical polymerizable monomer without an acid group,
at least one radical polymerizable monomer containing an acid group,
at least one fluoroaluminosilicate glass filler and/or radiopaque glass filler, and
at least one radical polymerization initiator,
wherein the fluoroaluminosilicate glass filler and/or the radiopaque glass filler is acid washed.

2. The composition according to claim 1,
wherein the at least one radiopaque glass filler comprises the following composition (wt. %): SiO₂: 20-80; B₂O₃: 2-15; BaO or SrO: 0-40; Al₂O₃: 2-20; CaO and/or MgO: 0-20; Na₂O, K₂O, Cs₂O: 0-10 each; WO₃: 0-20; ZnO: 0-20; La₂O₃: 0-10; ZrO₂: 0-15; P₂O₅: 0-30; Ta₂O₅, Nb₂O₅ or Yb₂O₃: 0-5; and CaF₂ and/or SrF₂ 0-10; and/or
the at least one fluoroaluminosilicate glass filler comprises the following composition (wt.-%): SiO₂: 20-35; Al₂O₃: 15-35; BaO or SrO: 10-25; CaO: 0-20; ZnO: 0-15; P₂O₅: 5-20; Na₂O, K₂O, Cs₂O: 0-10 each; and CaF₂: 0.5-20 wt. %;
wherein all figures are being based on the total mass of the glass, and all components except fluorine being calculated as oxides.

3. The composition according to claim 1,
wherein the fluoroaluminosilicate glass filler or radiopaque glass filler is washed with hydrochloric acid, nitric acid, formic acid, and/or acetic acid.

4. The composition according to to claim 1, which comprises
a) 10 to 80 wt. % of at least one fluoroaluminosilicate glass filler and/or radiopaque glass filler which is acid-washed,
b) optionally 0.1 to 25 wt. % of one or more further fillers,
c) 1 to 15 wt. % of at least one monomer containing an acid group,
d) 5 to 40 wt. % of at least one polyfunctional monomer without acid groups,
e) 0 to 10 wt. % of one or more oligomeric carboxylic acids,
f) 1 to 20 wt. % of one or more monofunctional monomers without acid groups,
g) 0.1 to 8 wt. % of an initiator for radical polymerization,
h) 0 to 20 wt. % water, and
i) 0.01 to 5 wt. % of one or more additives, in each case based on the total mass of the composition.

5. The composition according to claim 1,
wherein the at least one radically polymerizable monomer without an acid group comprises at least one polyfunctional monomer selected from bisphenol A-dimethacrylate, bis-GMA (an addition product of methacrylic acid and bisphenol A-diglycidyl ether), ethoxy- or propoxylated bisphenol-A-dimethacrylates, such as bisphenol-A-dimethacrylate with 3 ethoxy groups or 2,2-bis[4-(2-methacryloyloxypropoxy)phenyl]propane, UDMA (an addition product of 2-hydroxyethyl methacrylate and 2,2,4-trimethylhexamethylene-1,6-diisocyanate), tetramethylxylylene diurethane ethylene glycol di(meth)acrylate or tetramethylxylylene diurethane-2-methylethylene glycol diurethane di(meth)acrylate (V380), di-, tri- or tetraethylene glycol dimethacrylate, trimethylolpropane trimethacrylate, pentaerythritol tetramethacrylate, and glycerol di- and trimethacrylate, 1,4-butanediol dimethacrylate, 1,10-decanediol dimethacrylate (D₃MA), bis(methacryloyloxymethyl)tricyclo-[5.2.1.0^{2.6}]decane (DCP), polyethylene glycol or polypropylene glycol dimethacrylates, such as polyethylene glycol 200-dimethacrylate (PEG-200-DMA) or polyethylene glycol 400-dimethacrylate (-PEG-400-DMA), 1,12-dodecanediol dimethacrylate, urethanes of 2-(hydroxy-methyl)acrylic acid and diisocyanates, such as 2,2,4-trimethylhexamethylene diisocyanate or isophorone diisocyanate, pyrrolidones, such as 1,6-bis(3-vinyl-2-pyrrolidonyl)-hexane, bisacrylamides, such as methylene or ethylene bisacrylamide, bis(meth)acrylamides, such as N,N′-diethyl-1,3-bis(acrylamido)propane, 1,3-bis(methacrylamido)propane, 1,4-bis(acrylamido)butane or 1,4-bis(acryloyl)piperazine, and mixtures thereof.

6. The composition according to claim 1,
wherein the at least one radical polymerizable monomer containing an acid group comprises at least one monomer with a pKa of 0.5 to 4.0.

7. The composition according to claim 1,
wherein the at least one radical polymerizable monomer containing an acid group comprises at least one monomer selected from monomers containing a phosphoric ester group or phosphonic acid group, preferably 2-methacryloyloxyethylphenyl hydrogen phosphate, 10-methacryloyloxydecyl dihydrogen phosphate (MDP) glycerol dimethacrylate dihydrogen phosphate, dipentaerythritol pentamethacryloyloxyphosphate, 4-vinylbenzylphosphonic acid, 2-[4-(dihydroxyphosphoryl)-2-oxa-butyl]acrylic acid and/or 2-[4-(dihydroxyphosphoryl)-2-oxa-butyl]acrylic acid 2,4,6-trimethylphenyl ester, and/or 4-(meth)acryloyloxyethyltrimellitic anhydride, 10-methacryloyloxydecylmalonic acid, N-(2-hydroxy-3-methacryloyloxypropyl)-N-phenylglycine, and/or 4-vinylbenzoic acid.

8. The composition according to claim 4,
wherein the one or more oligomeric carboxylic acids comprises polyacrylic acid with a number average molecular weight of less than 7,200 g/mol.

9. The composition according to claim 1,
wherein the at least one radical polymerizable monomer without an acid group comprises at least one monofunctional monomer selected from benzyl, tetrahydrofurfuryl (meth)acrylate, isobornyl (meth)acrylate, p-cumyl-phenoxyethylene glycol methacrylate (CMP-1E) and 2-([1,1′-biphenyl]-2-oxy)ethyl methacrylate (MA-836), tricyclodecane methyl (meth)acrylate, 2-(2-biphenyloxy)ethyl (meth)acrylate, 2-hydroxyethyl (meth)acrylate, hydroxyethylpropyl (meth)acrylate, 2-acetoxyethyl methacrylate and mixtures thereof.

10. The composition according to claim 1, comprising a catalyst paste and a base paste, wherein the catalyst paste comprises
a) 10 to 80 wt. % of at least one acid-treated FAS and/or radiopaque glass filler,
b) 0.1 to 25 wt. % of one or more further fillers,
c) 2 to 30 wt. % of at least one monomer containing an acid group,
d) 5 to 40 wt. % of at least one polyfunctional monomer without acid groups,
e) 0 to 10 wt. % of one or more oligomeric carboxylic acids,
f) 1 to 20 wt. % of one or more monofunctional monomers,
g) 0.01 to 16 wt. % of at least one peroxide and/or hydroperoxide and optionally at least one photoinitiator,
h) 0 to 20 wt. % of water, and
i) 0.001 to 5 wt. % of one or more additives, in each case based on the total mass of the catalyst paste,
and where the base paste comprises
a) 10 to 80 wt. % of at least one acid-treated FAS and/or radiopaque glass filler,
b) 0.1 to 25 wt. % of one or more further fillers,
d) 5 to 40 wt. % of at least one polyfunctional monomer without acid groups,
f) 1 to 20 wt. % of one or more monofunctional monomers without acid groups,
g) 0.01 to 16 wt. % of at least one suitable reducing agent and optionally at least one photoinitiator,
h) 0 to 20 wt. % of water, and
i) 0.001 to 5 wt. % of one or more additives, in each case based on the total mass of the base paste.

11. The composition according to claim 1,
wherein the radical polymerizable composition is used therapeutically as a dental material selected from a dental cement, coating material, veneering material, restorative composite or luting cement.

12. The composition according to claim 1,
wherein the radical polymerizable composition is used non-therapeutically for preparation or repair of dental restorations selected from inlays, onlays, crowns, or bridges.

13. A method of treating a fluoroaluminosilicate glass filler or radiopaque glass filler with acid, wherein
(i) the fluoroaluminosilicate glass filler or radiopaque glass filler is dispersed in an aqueous solution of an organic and/or inorganic acid comprising hydrochloric acid, nitric acid, formic acid and/or acetic acid, the acid solution having an acid concentration of from 0.1 to 5 mol/l,
(ii) the dispersion is stirred for 0.5 to 24 h,
(iii) the filler is then separated and washed with deionized water, and
(iv) the filler is then separated and dried.

14. The process according to claim 13,
wherein, in step (iii), the filler is dispersed in deionized water and the dispersion is then stirred for 1 to 60 minutes, and this process is repeated 1 to 5 times.

15. A method of using the fluoroaluminosilicate glass filler or radiopaque glass filler preparable according to claim 13 for stabilizing dental radical polymerizable compositions.
